(19) United States
(12) Patent Application Publication
(10) Pub. No.: US 2024/0189057 A1
(43) Pub. Date: Jun. 13, 2024

(54) MAYO STAND ORGANIZER

(71) Applicant: Nabil Jamil Hamam, Dearborn Heights, MI (US)

(72) Inventor: Nabil Jamil Hamam, Dearborn Heights, MI (US)

(21) Appl. No.: 18/584,238

(22) Filed: Feb. 22, 2024

Related U.S. Application Data
(60) Provisional application No. 63/530,714, filed on Aug. 4, 2023.

(51) Int. Cl. A61B 50/20; A61B 46/10; A61B 50/15

(52) U.S. Cl. CPC A61B 50/20; A61B 46/10; A61B 50/15; A61B 2050/155

(57) ABSTRACT

The invention discloses a mayo stand organizer which comprises a cloth cover placed atop a mayo stand with four sides, positioned over a mayo stand cover, and further includes a first top layer with a first labelled zone located on the left side edge of the cloth cover and a box provided opposite to the first labelled zone; a second layer fitting snugly around the mayo stand using a first elastic band; and a third layer attached to the second layer through a second elastic band. The second layer comprises at least three separate rectangular shapes designed to accommodate surgical ties, and the third layer serves as a pocket for storing surgical instruments.

### FIELD OF THE INVENTION

The present invention relates generally to the field of surgical drapes which are adaptable to fit over and around a Mayo stand and in particular to a mayo stand organizer that improves and enhances efficiency and safety in an operating room.

### BACKGROUND OF THE INVENTION

A Mayo stand is a movable stand with a flat frame where surgical instruments can be held on a tray. This stand has a vertical support member with horizontal legs at the bottom. When viewed from the side, it resembles an open “C” shape, which allows the tray to fit over the lower end of a surgical table. The Mayo stand's legs are equipped with wheels to make it easy to move around the surgical table. The C-shaped design also enables the tray to be placed over the foot end of a patient or the surgical table, providing easy access to the nurse who can hand instruments to the operating surgeon directly from the tray on the Mayo stand.

An existing surgical drape designed for the Mayo stand consists of a long, tube-shaped bag that fits over both the horizontal tray and the vertical support section of the Mayo stand. This drape is comparable to a long pillowcase and is typically disposable and sterilized. The Mayo stand's tray usually has slightly raised edges, so when covered by the Mayo stand drape, it creates a sterile and covered surface. Surgical instruments required for the operation are placed on this tray by the nurse (by the Surgical Tech, or Scrub Nurse) in an orderly manner, including within the depressed surface of the tray. This organization ensures easy access for the surgical nurse to hand the instruments to the operating surgeon promptly when needed during the procedure.

Once the operating surgeon has finished using a specific instrument, they return it to the assisting nurse, (the Surgical Tech, or Scrub Nurse) in who then places it back on the tray for future use. However, it is common for the operating surgeon to instead place the used instrument on the patient's draped body rather than directly handing it to the nurse (the Surgical Tech, or Scrub Nurse) in. This approach allows the nurse (the Surgical Tech, or Scrub Nurse) in to replace the used surgical instruments during periods when she is not actively handing instruments to the operating surgeon.

U.S. Pat. No. 7,104,201B2 discloses a sterile device used to cover a surgical tray where surgical instruments are placed. The tray is covered by a thin, flat, elongated primary cover that is closed at one end and open at the other. The primary cover is placed over the tray and any supporting stand, creating the first layer of sterile protection. Additionally, there is a reinforced secondary cover placed over the primary cover and surgical tray, with the outer edges tucked under the tray's edges and pulled tightly together to secure and position the covers relative to the tray. This snug fit ensures that any excess material is gathered and secured, preventing it from interfering with the covers.

U.S. Pat. No. 5,170,804A discloses a disposable drape designed for a Mayo stand. The drape incorporates a tray that is also disposable and integrated into the design. The tray is divided into compartments to accommodate different shapes of surgical instruments. Additionally, one or more flaps are included near the edges of the tray. These flaps serve to cover the openings beneath the Mayo stand and the upper body of the patient, where surgical tools commonly get misplaced.

U.S. Pat. No. 8,739,797B2 discloses a draping system ensures a continuous sterile area between a patient's incision site and one or more medical practitioners. The drape incorporates a quick-release mechanism that allows a medical practitioner to detach from the sterile field without disturbing the sterility around them or the patient. The draping system may consist of a shortened practitioner gown, a drain, and an integrated patient incision area. Alternatively, it may include a flap that extends the continuous sterile field to one or more additional operating room tables.

U.S. Pat. No. 5,411,036A discloses a mayo stand cover is designed as a plastic tubing bag, and a specific section of the bag that covers the tray is narrowed down to create a snugger fit compared to the rest of the bag. This feature ensures that the bag fits smoothly on the tray and remains stable without easily shifting.

The main purpose of using a Mayo stand and drape is to offer a sterilized tray where surgical instruments can be systematically placed for easy access by the surgeon during the procedure. Despite the prior art Mayo-stand drape serving the fundamental needs of the operating surgeon effectively, it does come with certain drawbacks. Addressing these drawbacks would further enhance the efficiency and utility of a Mayo stand surgical drape. One significant disadvantage is the creation of a dead-air space or pocket beneath the drape near the patient's feet, which remains open on three sides. Unfortunately, this dead-air space becomes a cause for the loss of surgical instruments during the surgery.

Another drawback of the traditional Mayo-stand drape is that the standard steel tray lacks compartments, leading to inadequate separation and improper arrangement of the surgical instruments placed on it.

Accordingly, in view of the above-mentioned drawbacks, there is a need for an organizer for mayo stand.

### SUMMARY OF THE INVENTION

To address the foregoing problems, in whole or in part, and/or other problems that may have been observed by persons skilled in the art, the present disclosure provides compositions and methods as described by way of example as set forth below.

A principal object of the invention is to provide a mayo stand organizer that improves and enhances efficiency and safety in an operating room.

Another object of the invention is to provide a mayo stand organizer that offers additional protection and reduces the risk of surgical site infections and drape damage.

Another object of the invention is to provide a mayo stand organizer having a dedicated instrument holder improves efficiency by providing stability and preventing accidental falls during instrument transfers.

Another object of the invention is to provide a mayo stand organizer having a well-designed ties area that separates ties based on their sizes to prevent tangling.

In view of the foregoing, the present invention provides a mayo stand organizer which comprises a cloth cover placed atop a mayo stand with four sides, positioned over a mayo stand cover, and further includes a first top layer with a first labelled zone, (Neutral Zone) in located on the left side edge of the cloth cover and a box provided opposite to the first labelled zone; to standardize an area where for placement of sharps that prevents sharp injuries in the OR. a second layer fitting snugly around the mayo stand using a first elastic band or free without a band; and a third layer attached to the second layer through a second elastic band or sewed. The second layer comprises at least three separate rectangular shapes designed to accommodate surgical ties, and the third layer serves as a pocket for storing surgical instruments such as tie on a pass or passer (consists of a tie and clamp; this means one end of the tie is clamped in a hemostat or tonsil clamp and placed into the pocket of the third layer with the tip showing).

Additional features of the invention will be or will become apparent to one with skill in the art upon examination of the following figures and detailed description. It is intended that all such additional features and advantages be included within this description, be within the scope of the invention, and be protected by the accompanying claims.

### BRIEF DESCRIPTION OF THE DRAWINGS

Having thus described the subject matter of the present invention in general terms, reference will now be made to the accompanying drawings, which are not necessarily drawn to scale, and wherein:

FIG. 1 illustrates a schematic view of the mayo stand organizer, in accordance with an embodiment of the present invention;

FIG. 2 illustrates a schematic view of the mayo stand organizer with a first labelled Neutral zone and a box, in accordance with an embodiment of the present invention;

FIG. 3 illustrates a schematic view of the mayo stand organizer with a second layer and a third layer, in accordance with an embodiment of the present invention;

Skilled artisans will appreciate that elements in the drawings are illustrated for simplicity and may not have necessarily been drawn to scale. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having the benefit of the description herein.

### DETAILED DESCRIPTION OF THE INVENTION

The subject matter of the present invention now will be described more fully hereinafter with reference to the accompanying drawings, in which some, but not all embodiments of the subject matter of the present invention are shown. Like numbers refer to like elements throughout. The subject matter of the present invention may be embodied in many different forms and should not be construed as limited to the embodiments set forth herein; rather, these embodiments are provided so that this disclosure will satisfy applicable legal requirements. Indeed, many modifications and other embodiments of the subject matter of the present invention set forth herein will come to mind to one skilled in the art to which the subject matter of the present invention pertains having the benefit of the teachings presented in the foregoing descriptions and the associated drawings. All illustrations of the drawings are for the purpose of describing selected versions of the present invention and are not intended to limit the scope of the present invention. Therefore, it is to be understood that the subject matter of the present invention is not to be limited to the specific embodiments disclosed and that modifications and other embodiments are intended to be included within the scope of the appended claims.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and example of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one”, but does not exclude a plurality unless the contextual use dictates otherwise. When used herein to join a list of items, “of” denotes “at least one of the items”, but does not exclude a plurality of items of the list. Finally, when used herein to join a list of items, “and” denotes “all of the items of the list”.

The invention discloses a mayo stand organizer is composed of a cloth cover placed on top of a mayo stand, encompassing four sides and positioned over a mayo stand cover. Additionally, it consists of a first top layer with a designated zone on the left side edge of the cloth cover, and opposite to it, a box is provided where the surgical tech/scrub nurse tracking and documentation box is an inventive device designed for use during surgical procedures. It allows the user to track surgical sponges within the patient's cavity, preventing retention after closing. Additionally, it enables monitoring of local anesthesia and irrigation amounts for accurate Estimated Blood Loss (EBL) calculations. The box promotes standardized documentation practices, providing an easy-to-read and accessible solution while eliminating the need for disposable papers. This innovative device enhances surgical procedures, ensuring better record-keeping and contributing to a greener healthcare environment. The second layer fits snugly around the mayo stand, secured with a first elastic band, while the third layer is attached to the second layer through a second elastic band. Within the second layer, there are at least three separate rectangular shapes intended for accommodating surgical ties, while the third layer serves as a pocket designed to store surgical instruments.

The disclosed invention presents a Mayo Stand organizer, drape, and working area, introducing a three-layered, lint-free cloth cover specifically designed for the Mayo stand table in the operating room. This innovative organizer is equipped with a host of features that aim to significantly improve operating room efficiency and enhance patient safety. The three-layered cloth cover provides a superior level of protection by preventing the accumulation of lint and other contaminants on the Mayo stand table. This not only maintains the sterile environment but also reduces the risk of cross-contamination during surgical procedures, ultimately promoting better patient outcomes.

In an embodiment, the mayo stand organizer offers a well-thought-out working area, which includes designated spaces for efficient instrument organization. With a built-in instrument holder, the organizer ensures that surgical instruments remain securely in place, preventing accidental falls and streamlining the instrument transfer process. This feature allows nurses and surgical staff to focus more on patient care, ensuring smoother and more precise surgeries.

In an embodiment, one of the key highlights of the organizer is its integrated neutral zone, in alignment with safety recommendations. This neutral zone minimizes the risk of sharps-related injuries among the surgical team, providing a safer working environment during critical surgical tasks.

In an embodiment, the cloth cover features a dedicated ties area, cleverly designed to separate ties based on their sizes, eliminating tangling and confusion. Moreover, it incorporates a separate pocket under which surgical instruments loaded with ties can be conveniently placed. This intelligent design optimizes space utilization and allows for quick access to ties during surgeries, especially in situations requiring a prompt response to bleeding.

In an embodiment, addressing the challenge of retained sponges, the Mayo Stand organizer introduces a purpose-built tracking box within the cover. This tracking box facilitates systematic sponge counting, significantly reducing the likelihood of retained sponges and improving patient safety.

Overall, the innovative Mayo Stand organizer with its advanced features not only enhances operating room efficiency but also elevates the standard of patient safety during surgical procedures, making it a valuable and practical addition to modern healthcare facilities.

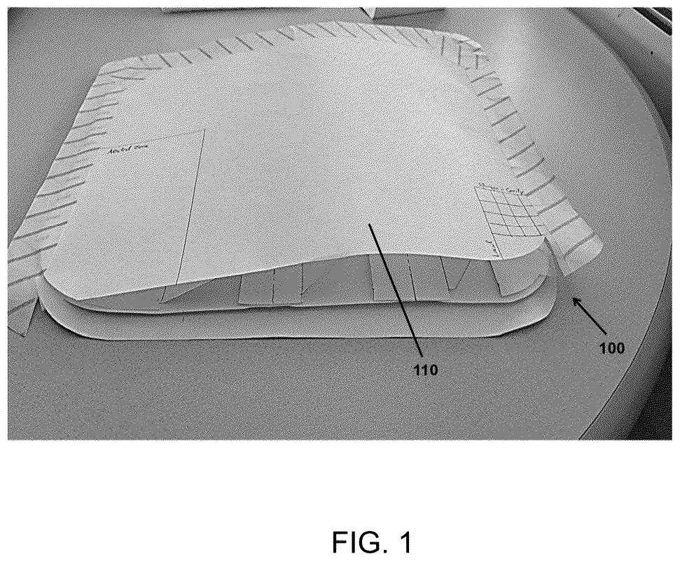

In accordance with an embodiment of the present invention, FIG. 1 illustrates a schematic view of the mayo stand organizer 100. The mayo stand organizer 100 is having a top layer 110 of the cover serves as a critical component of the innovative design, measuring 20.3×16 inches. Its versatility is enhanced by a small piece of velcro, providing convenient flipping capabilities whenever necessary during surgical procedures.

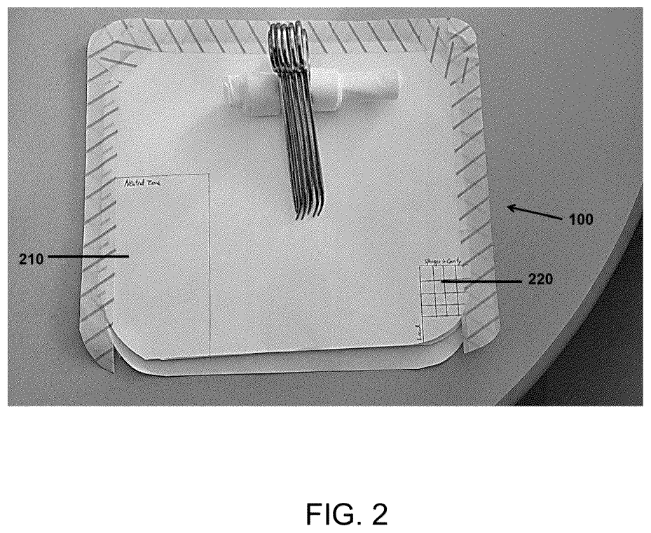

In accordance with an embodiment of the present invention, FIG. 2 illustrates a schematic view of the mayo stand organizer 100 with a first labelled Neutral zone 210 and a box 220. The top layer 110 is equipped with two strategically placed labels, imparting valuable functionality to the cover. On the left side, there is a rectangular label 210 measuring 8×5 inches, clearly indicating the “Neutral Zone.” Notably, this zone is specifically designed to be needlestick resistant, ensuring heightened safety for high-risk surgical procedures where the risk of sharps-related injuries is more prevalent. It could be with or without needle stick resistant.

Adjacent to the neutral zone label, the top layer 110 also features a small box 220 measuring 2.5×2.5 inches, which is aptly labelled “Sponges in.” Within this box 220, there are 10 small squares allotted for different purposes, including 1 square for local anesthesia and another square for irrigation. This well-organized layout allows for systematic sponge counting, minimizing the likelihood of retained sponges during surgeries and enhancing patient safety. The meticulous design and functional labels on the top layer 110 make it an indispensable asset in the operating room, facilitating seamless and efficient surgical procedures while prioritizing the well-being of both patients and the surgical team.

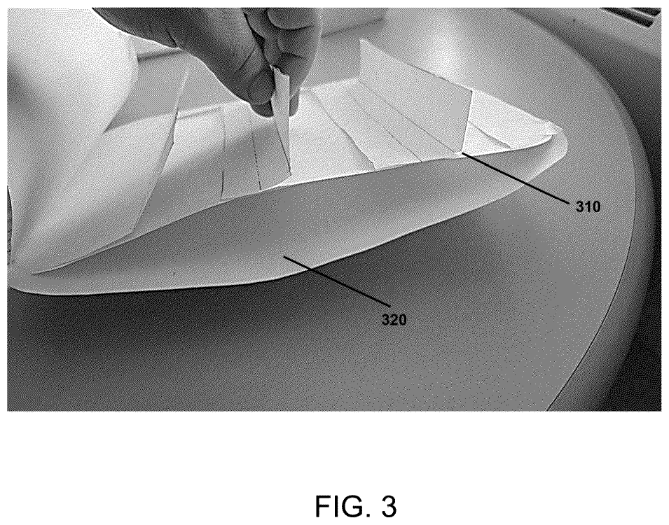

In accordance with an embodiment of the present invention, FIG. 3 illustrates a schematic view of the mayo stand organizer 100 with a second layer 310 and a third layer 320. There is provided a second layer 310 of the cover which plays a crucial role in the overall design, measuring 25×20 inches. It serves as a connecting element, securely attached to all three layers of the organizer. The clever integration of the second layer 310 ensures a snug fit around the Mayo stand table, achieved through an elastic band that provides a firm grip, preventing any slippage or movement during surgical procedures. This stable and secure placement is essential for maintaining a sterile environment and efficient workflow in the operating room.

Within this second layer 310, there are three separate rectangular shapes thoughtfully incorporated to accommodate surgical ties. These well-defined compartments allow for organized and systematic storage of various tie sizes, preventing tangling and facilitating quick access during surgeries. With easy visibility and accessibility, surgical staff can efficiently select the appropriate tie without any delays, ensuring smooth and uninterrupted surgical procedures. The careful inclusion of these rectangular shapes in the second layer 310 exemplifies the focus on optimizing the working area and enhancing surgical efficiency, making the Mayo Stand organizer 100 an invaluable tool in the operating room that streamlines surgical processes and contributes to better patient outcomes.

Further, as shown in figure, the third layer 320 of the cover is an essential component of the Mayo stand organizer 100, ingeniously attached to the second layer 310 using an elastic band. This innovative design allows for flexibility and adaptability, enabling the third layer 320 to be conveniently tucked under the table tray if needed. By utilizing this feature, surgical staff can adjust the setup according to their specific requirements, optimizing the workspace and ensuring a clutter-free environment during procedures.

The primary function of the third layer 320 is to serve as a large pocket, offering generous storage space for surgical instruments. With ample room available, surgical staff can easily access and store various instruments within arm's reach, streamlining the instrument-handling process and promoting efficient workflow in the operating room. The seamless integration of this pocket within the cover enhances organization and accessibility, reducing the time spent searching for specific instruments and ensuring a smooth and well-coordinated surgical operation. This user-friendly and practical aspect of the third layer 320 makes the Mayo Stand Organizer 100 an invaluable tool in the operating room, contributing to improved efficiency, reduced downtime, and ultimately better patient care.

In an embodiment, the mayo stand organizer 100 comprises an ingenious and specially designed instrument holder, constructed from a Spongey, or silicone, foam, cloth roll, or unique and high-quality material. This instrument holder plays a crucial role in the organization and accessibility of surgical instruments during procedures. The holder is thoughtfully equipped with rings or a C-shaped tool on each side, which can be easily moved either to the right or left, providing flexibility and adaptability to accommodate different instrument sizes and arrangements. This versatile feature ensures a secure hold on the instruments, preventing any unintended movements or displacement, and ultimately contributing to a safer and more efficient surgical environment.

Furthermore, to enhance stability and prevent accidental falls, the instrument holder is further equipped with small magnetic pieces located under the rings with magnetic or without. This magnetic feature enables the holder to firmly adhere to metal tables, maintaining a secure grip and eliminating any risks of instruments slipping or falling during critical surgical moments. The combination of the movable rings or C-shaped tool and the magnetic pieces in the instrument holder demonstrates the meticulous attention to detail in the design of the mayo stand organizer 100, prioritizing the safety and convenience of surgical staff.

The cover serves as a valuable addition to the mayo stand organizer 100, providing an extra layer of protection against potential punctures and contamination caused by sharp surgical instruments. By acting as a barrier between the instruments and the underlying surface, the cover effectively reduces the risk of surgical site infections and minimizes the chances of drape damage during procedures.

Further, by securely holding the surgical instruments in place, the instrument holder provides much-needed stability and prevents any accidental falls during instrument transfers. This ensures a smooth and seamless workflow in the operating room, allowing surgical techs, scrub nurses and surgical staff to concentrate their attention on providing optimal patient care without the distraction or concern of instrument mishandling.

The ties area of the cover efficiently separates ties based on their sizes, effectively preventing tangling and confusion during surgical procedures. This thoughtful organization not only saves valuable time for surgical staff but also ensures quick and easy access to the required tie without any delay. Moreover, the cover's ingenious design includes a separate pocket underneath, intended for placing surgical instruments that are loaded with ties. This feature optimizes the utilization of space on the mayo stand, providing a designated spot for instrument storage while keeping them easily accessible. This strategic arrangement allows for a swift response to bleeding situations, enhancing the overall efficiency and preparedness in the operating room and contributing to a smoother and more effective surgical process.

Some of the non-limiting advantages of the present invention are:
Additional layer of protection against punctures and contamination, reducing the risk of infections and drape damage
Minimizes the risk of sharps instrument related injuries, ensuring a safer surgical environment.
Provides stability and prevents accidental falls during instrument transfers, improving workflow and patient care.
Separates ties based on sizes, preventing tangling and optimizing space for quick response to bleeding situations.
Facilitates systematic sponge counting which reduces the risk of retained sponges and enhances patient safety.

Terms and phrases used in this document, and variations thereof, unless otherwise expressly stated, should be construed as open-ended as opposed to limiting. As examples of the foregoing: the term “including” should be read as mean “including, without limitation” or the like; the term “example” is used to provide exemplary instances of the item in the discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “of” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. Although the foregoing subject matter has been described in some detail by way of illustration and example for purposes of clarity of understanding, it will be understood by those skilled in the art that certain changes and modifications can be practiced within the scope of the appended claims.

What is claimed:

1. A mayo stand organizer, comprising:
a cloth cover placed atop a mayo stand, having four sides, wherein the cloth cover is positioned over a mayo stand cover; and further comprising
a first top layer having a first labelled Neutral zone on a left side edge of the cloth cover and a box is provided opposite to the first labelled zone;
a second layer having a snug fit around the mayo stand through a first elastic band; and
a third layer attached to the second layer through a second elastic band,
wherein the second layer includes at least three separate rectangular shapes to accommodate a surgical ties, and wherein the third layer provides a pocket to store surgical instruments.

2. The mayo stand organizer of claim 1, wherein the cloth cover is a three-layered lint-free cloth cover.

3. The mayo stand organizer of claim 1 comprises an instrument holder to securely hold the surgical instruments in place with a plurality of rings or a C-shaped tool positioned on each side of the instrument holder.

4. The mayo stand organizer of claim 1, wherein the plurality of rings or the C-shaped tool are movably coupled to the instrument holder to freely move in the left or right direction.

5. The mayo stand organizer of claim 1, wherein the plurality of rings or the C-shaped tool comprises a plurality of magnets to secure the instrument holder in place.

6. The mayo stand organizer of claim 1, wherein the first top layer comprises at least one velcro for flipping the first top layer.

7. The mayo stand organizer of claim 2, wherein the dimension of the top layer cover is 20.3×16 inches.

8. The mayo stand organizer of claim 1, wherein the dimension of the second layer cover is 25×20 inches.

9. The mayo stand organizer of claim 1, wherein the box positioned opposite to the first labelled zone comprises at least ten small squares, a local square and an irrigation square.

10. The mayo stand organizer of claim 1, wherein the dimension of the small box is 2.5×2.5 inches.

11. The mayo stand organizer of claim 1, wherein the first labelled zone of the first top layer is needlestick resistant for high-risk surgical procedures.

12. The mayo stand organizer of claim 1, wherein the cloth cover comprises a sponge tracking box to facilitate sponge counting.
